(19) United States
(12) Patent Application Publication
(10) Pub. No.: US 2009/0240323 A1
(43) Pub. Date: Sep. 24, 2009

(54) Controlled Degradation of Magnesium Stents

(75) Inventor: Josiah Wilcox, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/052,538

(22) Filed: Mar. 20, 2008

(51) Int. Cl. A61F 2/06

(52) U.S. Cl. 623/1.38; 623/1.42; 623/1.46; 427/2.25; 424/426

(57) ABSTRACT

Implantable medical devices, more specifically stents, are described herein comprising magnesium based core structures whose elimination times are slowed by the appropriate polymer coating. Appropriate biodegradable polymers are selected which are suitable to provide a specific degradation time for the magnesium based core structure. Bioactive agents are incorporated into the polymer coating in order to aid in the therapeutic effect of the stent.

### FIELD OF THE INVENTION

Medical devices are described herein comprising magnesium based core structures whose elimination times are controlled by the appropriate polymer coating. Appropriate biodegradable polymers are selected which are suitable to provide a slower elimination time for the magnesium based core structure.

### BACKGROUND OF THE INVENTION

Generally, implantable medical devices are intended to serve long term therapeutic applications and are not removed once implanted. In some cases it may be desirable to use implantable medical devices for short term therapies. Their removal, however, may require highly invasive surgical procedures that place the patient at risk for life threatening complications. It would be desirable to have medical devices designed for short term applications that degrade via normal metabolic pathways and are reabsorbed into the surrounding tissues.

Additionally, recent advances in in situ drug delivery have led to the development of implantable medical devices specifically designed to provide therapeutic compositions to remote anatomical locations. Perhaps one of the most exciting areas of in situ drug delivery is in the field of interventional cardiology. Vascular occlusions leading to ischemic heart disease are frequently treated using percutaneous transluminal coronary angioplasty (PTCA) whereby a dilation catheter is inserted through a femoral artery incision and directed to the site of the vascular occlusion. The catheter is dilated and the expanding catheter tip (the balloon) opens the occluded artery restoring vascular patency. Generally, a vascular stent is deployed at the treatment site to minimize vascular recoil and restenosis. In some cases, however, stent deployment leads to damage to the intimal lining of the artery which may result in vascular smooth muscle cell hyperproliferation and restenosis. When restenosis occurs it is necessary to either re-dilate the artery at the treatment site, or, if that is not possible, a surgical coronary artery bypass procedure must be performed.

Stents, useful for restoring and maintaining patency in biological lumens, can be manufactured from a variety of materials. These materials include, but are not limited to, metals and polymers. Both metal and polymer vascular stents have been associated with thrombosis and chronic inflammation at the implantation site and impaired remodeling at the stent site. It has been proposed that limiting the exposure of the vessel to the stent to the immediate intervention period would reduce late thrombosis and chronic inflammation. One means to produce a temporary stent is to implant a bioabsorbable, or biodegradable, stent.

There are several parameters to consider in the selection of a bioabsorbable material for stent manufacture. These include, but are not limited to, the strength of the material to avoid potential immediate recoil, the rate of degradation and corrosion, biocompatibility with the vessel wall and lack of toxicity. Additionally, it may be desirable to include bioactive agents in the bioabsorbable stent such that the bioactive agent is release at the implantation site during degradation of the stent. The mechanical properties and release profiles of bioactive agents directly depend on the rate of degradation of the stent material which is controlled by selection of the stent materials, passivation agents and the manufacturing process of the stent. Currently there are two types of materials used in bioabsorbable stents, polymers and metals.

Metal bioabsorbable stents are attractive since they have the potential to perform similarly to stainless steel metal stents. One such material is magnesium and bioresorbable magnesium alloy stents have been shown to induce less thrombosis in damaged arteries than conventional bare metal stents.

Stents that have sufficient strength to hold the artery open and then dissolve in short periods of time, less than twelve months, are considered desirable. Current degradable stents use a polymer based construction that takes longer than one year to degrade and requires large thick struts which limit deliverability. Magnesium based stents have been shown to have acceptable crossing profiles (e.g. balloon expandable) and radial strength but bare magnesium has been shown to degrade too rapidly to be useful in arterial remodeling. Current magnesium based stents have a degradation time of about one month. Such magnesium stents which degrade is one month or less have been shown to be subject to constructive vascular remodeling. Constructive vascular remodeling has contributed to late loss and target lesion revascularization. For a degradable stent to be considered desirable, it will have to maintain a sufficient radial strength for greater than one month.

Therefore, there exists a need for a bioabsorbable stent which incorporates the strength (e.g. radial strength) characteristics of a metal stent, the drug eluting properties of a polymer based stent and a desirable controlled degradation time.

### SUMMARY OF THE INVENTION

Implantable medical devices, more specifically stents, are described herein comprising magnesium based core structures whose degradation times are controlled by an appropriate polymer coating. Appropriate biodegradable polymers are selected which are suitable to provide a specific degradation time for the magnesium based core structure. Bioactive agents are incorporated into the polymer coating in order to aid in the therapeutic effect of the stent.

Described herein are stents comprising a magnesium based core structure, the core structure having a degradation time; a polymeric material associated with the core structure, the polymeric material having an ability to slow the degradation time; and a bioactive agent associated with the polymeric material.

Described herein are stents comprising: (a) a magnesium based core structure, the core structure having a first degradation time; (b) at least one polymeric material coated on at least a portion of the core structure, said polymeric material having an ability to slow said degradation time such that said polymeric material coated on at least a portion of the core has a second degradation time; and (c) at least one bioactive agent associated with the at least one polymeric material. In one embodiment, the stent is selected from the group consisting of woven stents, individual ring stents, sequential ring stents, closed cell stents, open cell stents, laser cut tube stents, ratchet stents, and modular stents.

In one embodiment, the magnesium based core structure comprises magnesium and magnesium alloys. In another embodiment, the second degradation time is between 1 month and 12 months.

In one embodiment, the polymeric material comprises a top coat. In one embodiment, the at least one polymeric material comprises polymers selected from the group consisting of polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactone polymers, polytrimethylene carbonate, polyacrylates, polyethylene glycol, hydrogels, photo-curable hydrogels, terminal diols, and combinations thereof.

In one embodiment, the at least one bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578). In another embodiment, the at least one bioactive agent is coated on said polymeric material. In another embodiment, the at least one bioactive agent is dispersed within said polymer material.

Described herein is a method of prolonging the life of an implantable magnesium based medical device comprising: (a) providing a magnesium based core structure comprising a first degradation time; (b) choosing at least one appropriate bioabsorbable polymeric material; (c) coating at least a portion of the core structure with the polymeric material forming a coated medical device, thereby retarding the degradation of the core structure; and (d) providing a medical device having a second degradation time. In one embodiment, the magnesium based core structure comprises magnesium and magnesium alloys.

In one embodiment, the first degradation time is less than 1 month. In another embodiment, the second degradation time is between 1 month and 12 months.

In one embodiment, the at least one polymeric material is bioabsorbable and comprises polymers selected from the group consisting of polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactone polymers, polytrimethylene carbonate, polyacrylates, polyethylene glycol, hydrogels, photo-curable hydrogels, terminal diols, and combinations thereof. In another embodiment, the at least one polymeric material is a top coat.

In one embodiment, the bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578). In another embodiment, the bioactive agent is coated on the at least one polymeric material. In another embodiment, the bioactive agent is dispersed within the at least one polymer material.

In one embodiment, the implantable medical device is selected from the group consisting of woven stents, individual ring stents, sequential ring stents, closed cell stents, open cell stents, laser cut tube stents, ratchet stents, and modular stents.

### Definition of Terms

Before proceeding it may be useful to define many of the terms used to describe the present invention. Words and terms of art used herein should be first defined as provided for in this specification, and then as needed as one skilled in the art would ordinarily define the terms.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Bioabsorbable: As used herein “bioabsorbable” refers to a material that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however all bioabsorbable polymers are considered biodegradable.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein as first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Compatible: As used herein “compatible” refers to a composition posing the optimum, or near optimum combination of physical, chemical, biological and drug release kinetic properties suitable for a controlled-release coating made in accordance with the teachings of the present invention. Physical characteristics include durability and elasticity/ductility, chemical characteristics include solubility and/or miscibility and biological characteristics include biocompatibility. The drug release kinetic should be either near zero-order or a combination of first and zero-order kinetics.

Delayed Release: As used herein “delayed release” refers to the release of bioactive agent(s) after a period of time and/or after an event or series of events.

Drug or bioactive agent: As used herein “drug” or “bioactive agent” shall include any agent having a therapeutic effect in an animal. Exemplary, non limiting examples include anti-proliferatives including, but not limited to, macrolide antibiotics including FKBP 12 binding compounds, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids, cytostatic compounds, toxic compounds, anti-inflammatory compounds, chemotherapeutic agents, analgesics, antibiotics, protease inhibitors, statins, nucleic acids, polypeptides, and delivery vectors including recombinant micro-organisms, liposomes, the like.

### DETAILED DESCRIPTION OF THE INVENTION

Medical devices are described herein comprising magnesium based core structures whose degradation times can be controlled by an appropriate polymer coating. Appropriate bioabsorbable polymers can be selected which are suitable to provide a slower degradation time for the magnesium based core structure. The bioabsorbable polymers can also be used as a means of controlled release of a bioactive agent.

In one embodiment, the implantable medical device is a stent. The stent architectures suitable for fabrication are not limited to the examples provided herein but can include coil stents, helical spiral stents, woven stents, individual ring stents, sequential ring stents, closed cell stents, open cell stents, laser cut tube stents, ratcheting stents, modular stents and the like. Additionally, stents adapted for deployment in any vessel or duct to maintain patency including, but not limited to vascular stents, stent grafts, biliary stents, esophageal stents, and stents of the trachea or large bronchi, ureters, and urethra are also consider within the scope of the present description.

In one embodiment, the stents comprise a magnesium based core. Magnesium and its alloys are biocompatible, bioabsorbable and easy to mechanically manipulate presenting an attractive solution for bioabsorbable stents. Radiological advantages of magnesium include compatibility with magnetic resonance imaging (MRI), magnetic resonance angiography and computed tomography (CT). Vascular stents comprising magnesium and its alloys are less thrombogenic than other bare metal stents. The biocompatibility of magnesium and its alloys stems from its relative non-toxicity to cells. Magnesium is abundant in tissues of animals and plants; specifically, magnesium is the fourth most abundant metal ion in cells, the most abundant free divalent ion and therefore is deeply and intrinsically woven into cellular metabolism. Magnesium-dependent enzymes appear in virtually every metabolic pathway is also used as a signaling molecule.

Magnesium alloys suitable for bioabsorbable stents include alloys of magnesium with other metals including, but not limited to, aluminum and zinc. In one embodiment, the magnesium alloy comprises between about 1% and about 10% aluminum and between about 0.5% and about 5% zinc.

The magnesium alloys can include but are not limited to Sumitomo Electronic Industries (SEI, Osaka, Japan) magnesium alloys AZ31 (3% aluminum, 1% zinc and 96% magnesium) and AZ61 (6% aluminum, 1% zinc and 93% magnesium). The desirable features of the alloy include high tensile strength and responsive ductility. Tensile strength of typical AZ31 alloy is at least 280 MPa while that of AZ61 alloy is at least 330 MPa.

In order to increase the degradation time of the magnesium based cores of the stents described herein, bioabsorbable polymers can be coated onto at least a portion of the stent. Suitable bioabsorbable polymers include, but are not limited to, polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactone polymers, polytrimethylene carbonate, polyacrylates, polyethylene glycol, hydrogels, photo-curable hydrogels, terminal diols, co-polymers of 2 or more of the above and combinations thereof.

Varying the monomer ratios allows the ordinarily skilled artisan to fine tune, or to modify, the properties of the polymer. The properties of bioabsorbable polymers arise from the monomers used and the reaction conditions employed in their synthesis including but not limited to, temperature, solvents, reaction time and catalyst choice. In order to tune, or modify, the polymers, a variety of properties are considered including, but not limited to, T_{g}, connectivity, molecular weight, thermal properties, and degradation time.

Fine tuning, or modifying, the glass transition temperature (T_{g}) of the bioabsorbable polymers is also taken into account. Bioactive agent elution from polymers depends on many factors including density, the bioactive agent to be eluted, molecular composition of the polymer and T_{g}. Higher T_{g}s, for example temperatures above 40° C., result in more brittle polymers while lower T_{g}s, e.g lower than 40° C., result in more pliable and elastic polymers at higher temperatures. Bioactive agent elution is slow from polymers that have high T_{g}s while faster rates of bioactive agent elution are observed with polymers possessing low T_{g}s. In one embodiment, the T_{g} of the polymer is selected to be lower than 37° C.

Polymers used for coating having relatively high T_{g}s can result in medical devices with unsuitable drug eluting properties as well as unwanted brittleness. In the cases of polymer-coated vascular stents, a relatively low T_{g} in the coating polymer effects the deployment of the vascular stent. For example, polymer coatings with low T_{g}s are “sticky” and adhere to the balloon used to expand the vascular stent during deployment, causing problems with the deployment of the stent. Low T_{g} polymers, however, have beneficial features in that polymers having low T_{g}s are more elastic at a given temperature than polymers having higher T_{g}s. Expanding and contracting a polymer-coated vascular stent mechanically stresses the coating. If the coating is too brittle, i.e. has a relatively high T_{g}, then fractures may result in the coating possibly rendering the coating inoperable. If the coating is elastic, i.e has a relatively low T_{g}, then the stresses experienced by the coating are less likely to mechanically alter the structural integrity of the coating. Therefore, the T_{g}s of the polymers can be fine tuned for appropriate coating applications by a combination of monomer composition and synthesis conditions. The polymers are engineered to have adjustable physical properties enabling the practitioner to choose the appropriate polymer for the function desired.

Medical devices, including implantable medical devices, are coated with the polymers disclosed herein and therefore the physical properties of the polymers are considered in light of the specific application at hand. Physical properties of the polymers can be fine tuned so that the polymers can optimally perform for their intended use. Properties that can be fine tuned, without limitation, include T_{g}, molecular weight (both Mₙ and M_{w}), polydispersity index (PDI, the quotient of M_{w}/Mₙ), degree of elasticity and degree of amphiphlicity. In one embodiment, the T_{g} of the polymers range from about −10° C. to about 85° C. In still another embodiment, the PDI of the polymers range from about 1.35 to about 4. In another embodiment, the T_{g} of the polymers ranges form about 0° C. to about 40° C. In still another embodiment, the PDI of the polymers range from about 1.5 to about 2.5.

Different polymers used to coat medical devices can have different degradation times in a cardiovascular (in vivo) environment. Functional groups, methods of polymer coordination, catalysts, polymer molecular weight, and hydrophobicity can all be relied upon to develop a polymer for coating onto an implantable medical device that has a tailored degradation time.

In one embodiment, the polymers can be applied to the magnesium based core as a top coat. As a top coat, the polymers restrict the body fluids, enzymes and cells from degrading the magnesium core. As a result, the degradation time of the stent can be extended by at least the time required to degrade the polymer.

In another embodiment, multiple polymeric layers can be applied to the magnesium based core. The other most layer can be considered the top coat. In such an embodiment, for example, polymers can be utilized that will be most compatible with the surrounding tissue as the surrounding tissue develops around the stent. For example, a polymer that aids in supporting the radial strength of the stent may be used as a first coat and thereon are layered one or more additional polymer coatings that are more biocompatible.

Degradation times for bare magnesium stents are about one month. In one embodiment, the first degradation time of the bare magnesium stent can be increased by application of a polymeric coating on the stent, the polymeric material having a second degradation time longer than that of the first degradation time. The over all degradation time of the polymeric material and the magnesium stent is thereby increased to a new degradation time longer than that of the two separately. In one embodiment, the new degradation time is less than 6 months. In another embodiment, the new degradation time is less than 12 months. In another embodiment, the new degradation time is less than 9 months. In another embodiment, the new degradation time is between about 1 month and about 3 months. In another embodiment, the new degradation time is between about 1 month and about 6 months. In another embodiment, the new degradation time is between about 1 month and about 9 months. In another embodiment, the new degradation time is between about 1 month and about 12 months. In another embodiment, the new degradation time is between about 3 months and about 9 months. In another embodiment, the new degradation time is between about 6 months and about 9 months. In another embodiment, the new degradation time is between about 3 months and about 12 months. In another embodiment, the new degradation time is between about 6 months and about 12 months.

In another embodiment, only selected portions of the magnesium core are coated. In such a scenario, only selected portions of the core that are coated have an increased degradation time. The remaining portions of the magnesium core which are uncoated will degrade at the normal rate of a bare magnesium stent.

In another embodiment, different regions of the magnesium core can be coated with different polymer combinations. In such a scenario, different portions of the magnesium core can be tailored to degrade at different rates which are dependent on the polymer used to coat that specific portion. In such an embodiment, limitless combinations of coatings can be applied to the magnesium core.

The bioabsorbable magnesium stents of the present invention are also useful for the delivery and controlled release of bioactive agents. Bioactive agents that are suitable for release from the stents include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

The polymeric materials discussed herein may be designed to provide local delivery of a specific dose of bioactive agent. That dose may be a specific weight of bioactive agent added or a bioactive agent to polymer ratio. In one embodiment, the medical device can be loaded with 0 to 1000 μg of bioactive agent; in another embodiment, 5 μg to 500 μg; in another embodiment 10 μg to 250 μg; in another embodiment, 15 μg 150 μg. A ratio may also be established to describe how much bioactive agent is added to the polymer that is coated to the medical device. In one embodiment a ratio of 1 part bioactive agent:1 part polymer may be used; in another embodiment, 1:1-5; in another embodiment, 1:1-9; in another embodiment, 1:1-20.

Exemplary, non limiting examples of bioactive agents include anti-proliferatives including, but not limited to, macrolide antibiotics including FKBP-12 binding compounds, mTOR inhibitors estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers described herein.

In addition to the site specific delivery of bioactive agent, the implantable medical devices discussed herein can accommodate one or more additional bioactive agents. The choice of bioactive agent to incorporate, or how much to incorporate, will have a great deal to do with the polymer selected to coat or form the implantable medical device. A person skilled in the art will appreciate that hydrophobic agents are generally attracted to hydrophobic polymers and hydrophilic agents are generally attracted to hydrophilic polymers. In one embodiment, the polymeric coating is hydrophilic and the bioactive agent is hydrophilic. In another embodiment, the polymeric coating is hydrophobic and the bioactive agent is hydrophobic.

In one embodiment, the polymer coating can comprise a mixture of hydrophilic and hydrophobic polymers or a polymeric material comprising a mixture of hydrophobic and hydrophilic monomers. In one embodiment, a blend of hydrophobic and hydrophilic polymers is coated onto the medical device. A blend coating such as this can exhibit properties such as, but not limited to, a hydrophobic core to accommodate hydrophobic bioactive agents and a hydrophilic surface to increase the biocompatibility of the coated device.

In one embodiment, the bioactive agent is covalently bonded to the bioabsorbable polymer. The covalently-bound bioactive agent is released in situ from the degrading polymer with the polymer degradation products thereby ensuring a controlled bioactive agent supply throughout the degradation course. The bioactive agent is released to the treatment site as the polymeric material is exposed through biodegradation.

In another embodiment, the bioactive agent is contained within pores or reservoirs within the bioabsorbable polymer and is released in situ from the degrading polymer thereby ensuring a controlled bioactive agent supply throughout the degradation course.

In one embodiment, multiple polymeric layers can be coated on the magnesium stent core. At least one of the polymeric layers can contain a bioactive agent. Bioactive agents can be coated with appropriate polymers to increase or decrease their respective elution times from the stent. Layers can be used on top of the bioactive agent containing polymer layers to retard the delivery of the bioactive agent even further or even block it from being delivered for a predetermined times based on the polymer or polymers used.

In one embodiment, one or more polymeric layers which contain one or more bioactive agents can be coated on the magnesium core. Coated on top can be one or more polymeric layers used to extent stent degradation time. In another embodiment, one or more polymeric layers which can be used to extent stent degradation time can be coated on the magnesium core. Coated on top can be coated one or more polymeric layers which contain one or more bioactive agents.

### EXAMPLE 1

### Metal Stent Cleaning Procedure

Magnesium stents are placed in a glass beaker and covered with reagent grade or better hexane. The beaker containing the hexane immersed stents is then placed into an ultrasonic water bath and treated for 15 minutes at a frequency of between approximately 25 to 50 KHz. Next the stents are removed from the hexane and the hexane is discarded. The stents are then immersed in reagent grade or better 2-propanol and vessel containing the stents and the 2-propanol is treated in an ultrasonic water bath as before. Following cleaning, the stents with organic solvents are thoroughly washed with distilled water and thereafter immersed in 1.0 N sodium hydroxide solution and treated at in an ultrasonic water bath as before. Finally, the stents are removed from the sodium hydroxide, thoroughly rinsed in distilled water and then dried in a vacuum oven over night at 40° C. After cooling the dried stents to room temperature in a desiccated environment they are weighed their weights are recorded.

### EXAMPLE 2

### Coating a Clean, Dried Stent

In the following Example, ethanol is chosen as the solvent of choice; the polymer is soluble in tetrahydrofuran (THF). Persons having ordinary skill in the art of polymer chemistry can easily pair the appropriate solvent system to the polymer and achieve optimum results with no more than routine experimentation.

250 mg of polycaprolactone (PCL) is added to the 2.8 mL of THF and mixed until the PCL is dissolved and a polymer solution is generated.

The cleaned, dried stents are coated using either spraying techniques or dipped into the polymer solution. The stents are coated as necessary to achieve a final coating weight of between approximately 10 μg to 1 mg. Finally, the coated stents are dried in a vacuum oven at 50° C. over night. The dried, coated stents are weighed and the weights recorded. The resulting polymer coating can have a degradation time of about 3 months.

### EXAMPLE 3

### Coating a Clean, Dried Stent

250 mg of poly-D-lactide (PDL) is added to the 2.8 mL of THF and mixed until the PDL is dissolved and a polymer solution is generated.

The cleaned, dried stents are coated using either spraying techniques or dipped into the polymer solution. The stents are coated as necessary to achieve a final coating weight of between approximately 10 μg to 1 mg. Finally, the coated stents are dried in a vacuum oven at 50° C. over night. The dried, coated stents are weighed and the weights recorded. The resulting polymer coating can have a degradation time of about 6 months.

### EXAMPLE 4

A stent can be coated first with the polymeric coating described in Example 2 and then by the polymeric material described in Example 3. The two polymeric layers can have a combined degradation time of about 9 months.

### EXAMPLE 5

A stent with a polymeric coating according to Example 2 can further include a bioactive agent dispersed within the polymer to be coated. For example, an mTOR inhibitor can be added to the polymeric material to be coated. The stent can be dipped into the polymeric material/bioactive agent blend thereby coating the blend onto the stent.

### EXAMPLE 6

A stent with a polymeric coating according to Example 3 can further include a bioactive agent dispersed within the polymer to be coated. For example, an mTOR inhibitor can be added to the polymeric material to be coated. The stent can be dipped into the polymeric material/bioactive agent blend thereby coating the blend onto the stent.

### EXAMPLE 7

A stent as described in Example 5 can be further dipped into a polymeric material/bioactive agent blend of Example 6. The resulting stent will have a combined degradation time of at least 9 months and can elute an mTOR inhibitor from both coatings.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A stent comprising:
(a) a magnesium based core structure, said core structure having a first degradation time;
(b) at least one polymeric material coated on at least a portion of said core structure, said polymeric material having an ability to slow said degradation time such that said polymeric material coated on at least a portion of said core has a second degradation time; and
(c) at least one bioactive agent associated with said at least one polymeric material.

2. The stent according to claim 1 wherein said stent is selected from the group consisting of woven stents, individual ring stents, sequential ring stents, closed cell stents, open cell stents, laser cut tube stents, ratchet stents, and modular stents.

3. The stent according to claim 1 wherein said magnesium based core structure comprises magnesium and magnesium alloys.

4. The stent according to claim 1 wherein said second degradation time is between 1 month and 12 months.

5. The stent according to claim 1 wherein said polymeric material comprises a top coat.

6. The stent according to claim 1 wherein said at least one polymeric material comprises polymers selected from the group consisting of polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactone polymers, polytrimethylene carbonate, polyacrylates, polyethylene glycol, hydrogels, photo-curable hydrogels, terminal diols, and combinations thereof.

7. The stent according to claim 1 wherein said at least one bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578).

8. The stent according to claim 1 wherein said at least one bioactive agent is coated on said polymeric material.

9. The stent according to claim 1 wherein said at least one bioactive agent is dispersed within said polymer material.

10. A method of prolonging the life of an implantable magnesium based medical device comprising:
(a) providing a magnesium based core structure comprising a first degradation time;
(b) choosing at least one appropriate bioabsorbable polymeric material;
(c) coating at least a portion of said core structure with said polymeric material forming a coated medical device, thereby retarding the degradation of said core structure; and
(d) providing a medical device having a second degradation time.

11. The method according to claim 10 wherein said magnesium based core structure comprises magnesium and magnesium alloys.

12. The method according to claim 10 wherein said first degradation time is less than 1 month.

13. The method according to claim 11 wherein said second degradation time is between 1 month and 12 months.

14. The method according to claim 11 wherein said at least one polymeric material is bioabsorbable and comprises polymers selected from the group consisting of polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactone polymers, polytrimethylene carbonate, polyacrylates, polyethylene glycol, hydrogels, photo-curable hydrogels, terminal diols, and combinations thereof.

15. The method according to claim 11 wherein said at least one polymeric material is a top coat.

16. The method according to claim 11 wherein said bioactive agent is selected from the group consisting of anti-proliferatives, mTOR inhibitors, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, transforming nucleic acids, sirolimus (rapamycin), tacrolimus (FK506), everolimus (certican), temsirolimus (CCI-779) and zotarolimus (ABT-578).

17. The method according to claim 11 wherein said bioactive agent is coated on said at least one polymeric material.

18. The method according to claim 11 wherein said bioactive agent is dispersed within said at least one polymer material.

19. The method according to claim 11 wherein said implantable medical device is selected from the group consisting of woven stents, individual ring stents, sequential ring stents, closed cell stents, open cell stents, laser cut tube stents, ratchet stents, and modular stents.
